(19) United States
(12) Patent Application Publication
(10) Pub. No.: US 2006/0184064 A1
(43) Pub. Date: Aug. 17, 2006

(54) Urine sample collection device

(75) Inventors: Robert W. Paasch, Aumsville, OR (US); Michael J. Woodward, Aumsville, OR (US); Anthony Michael Arnautov, Wilsonville, OR (US)

Correspondence Address: MARGER JOHNSON & MCCOLLOM, P.C., 210 SW MORRISON STREET, SUITE 400, PORTLAND, OR, 97204, US

(73) Assignee: SAMPLE RITE, INC., Aumsville, OR (US)

(21) Appl. No.: 11/329,918

(22) Filed: Jan. 10, 2006

Related U.S. Application Data
(63) Continuation-in-part of application No. 10/825,615, filed on Apr. 14, 2004, now Pat. No. 7,011,634.
(60) Provisional application No. 60/462,986, filed on Apr. 14, 2003.

(51) Int. Cl. A61B 5/00; A47K 11/00; A61M 1/00

(52) U.S. Cl. 600/573; 600/574; 604/317; 4/144.1

(57) ABSTRACT

A urine collection device includes a support apparatus for supporting the urine collection device on a toilet. An attachment section attaches to a sample holder and a collection section receives and directs urine into the sample holder.

### CROSS REFERENCE TO RELATED APPLICATIONS

This application is a continuation-in-part of U.S. patent application Ser. No. 10/825,615, filed on Apr. 14, 2004, now pending, which is herein incorporated by reference in its entirety, which claims the benefit of U.S. Provisional Application No. 60/462,986, filed on Apr. 14, 2003, now expired.

### BACKGROUND

It is difficult for many people to hold a urine sample cup effectively to catch their own urine stream flow. It is even more difficult for the elderly, disabled, overweight, pregnant and very young to give urine samples. It is not easy or sanitary for anyone. Trying to catch a “mid-stream” flow multiplies the difficulties. A mid-stream flow refers to a urine sample that does not include the initial portion of a urine stream. In other words, a urine sample taken after the patient has already started urinating. Mid-stream urine samples can be important, since the initial portion of the urine stream may be contaminated.

When patients or sample-givers are faced with the indignity of someone else holding the urine sample cup for them, it sometimes becomes impossible for the patient to pass urine on demand. It is also time consuming, unsanitary and difficult for the nurses.

A “nurse's hat” is a large plastic disposable pot that looks like an upside down hat in which the brim is the funnel and the bowl collects the urine flow. After the urine sample is deposited into the nurse's hat, the nurse pours a portion of the urine into a sample cup. This is time consuming and unsanitary for the nurses and patients. The nurse's hat also becomes a medical waste disposal issue because it is large and bulky and creates a large volume of waste.

The present invention addresses this and other problems associated with the prior art.

### SUMMARY OF THE INVENTION

A support apparatus supports a urine collection device on a toilet. An attachment section attaches to a sample holder and a collection section receives and directs urine into the sample holder. The urine collection device allows most people to collect their own urine sample easily and effectively. The sample device has the advantage of collecting a “mid-stream” urine flow thus eliminating having to pour a urine sample into a sample cup. The device is easy to use, economical to manufacture, and allows people to give a urine sample in privacy.

The foregoing and other objects, features and advantages of the invention will become more readily apparent from the following detailed description of a preferred embodiment of the invention which proceeds with reference to the accompanying drawings.

### BRIEF DESCRIPTION OF THE DRAWINGS

FIG. 1 is a perspective view of a first embodiment of the Urine Sample Collection Device (USCD) installed on a toilet.

FIG. 2 is perspective isolation view of the USCD shown in FIG. 1.

FIG. 3 is a side-sectional view of the USCD shown in FIG. 1.

FIG. 4 is a perspective view of a second embodiment of the USCD installed on a toilet.

FIG. 5 is a perspective isolation view of the USCD shown in FIG. 4.

FIG. 6 is a side-sectional view of the USCD shown in FIG. 4.

FIG. 7 is a perspective isolation view of a third embodiment of the USCD.

FIG. 8 is a side-sectional view of the USCD shown in FIG. 7.

FIG. 9 is a front isolation view of the USCD shown in FIG. 7.

### DETAILED DESCRIPTION

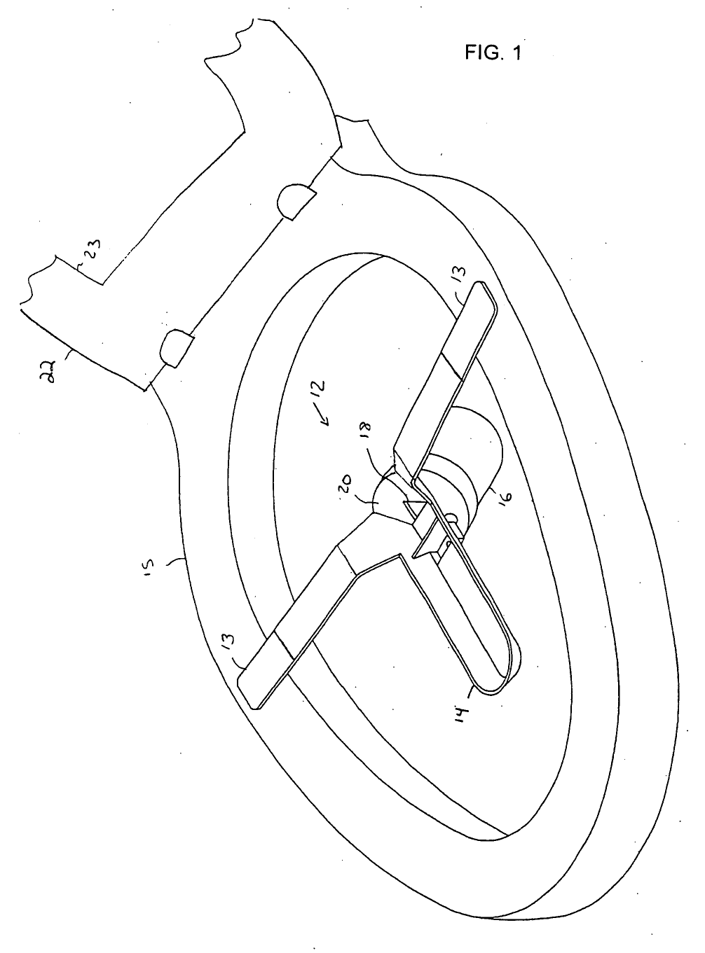

Referring to FIG. 1, the Urine Sample Collection Device (USCD) 12 is a hands-free, self-supporting urine sample funneling and mid-stream collecting system for male or female people. The USCD 12 includes support arms 13 that support the USCD 12 on top of a toilet bowl 15. A collection section 14 collects urine from a user and funnels the urine toward an opening 18 in a sample cup attachment section 20. A urine sample cup 16 is attached underneath section 20 for retaining the urine funneled down from collection section 14.

The USCD 12 collects the urine sample by first being placed on the toilet bowl 15 or on top of the toilet seat 22. The thinness and narrow width of the support arms 13 allow the USCD 12 to be placed on top of the toilet bowl 14 and the toilet seat 22 placed down on top of the USCD 12 and bowl 14. The USCD 12 is secured in position by the weight of the user sitting on top of the toilet seat 22. In an alternative embodiment, the USCD 12 is placed across the ring 23 of the toilet seat 22.

In the embodiment shown the sample cup 16 is disposed at approximately a 45-degree downward angle with respect to a horizontal defined by the toilet seat 22. The collection section 14 is disposed at approximately a 15-degree upward angle with respect to the horizontal. Advantageously, angling the sample cup 16 prevents the sample cup from filling too high. Filling a sample cup 16 too increases the likelihood of a spill during transportation of the sample cup. Also advantageously, angling the sample cup 16 decreases the chance of the sample cup 16 contacting toilet water or a toilet bowl when the USCD 12 is used in variously sized toilets.

The USCD 12 is placed generally in the middle of the toilet bowl 15 or toilet seat 22 with the collection section 14 being directed forward toward a front end of the toilet bowl 15. The collection section 14 thereby is positioned directly under the urinary track of the user. This allows a person providing a urine sample to sit down on the toilet seat 22 and pass urine in a normal manner.

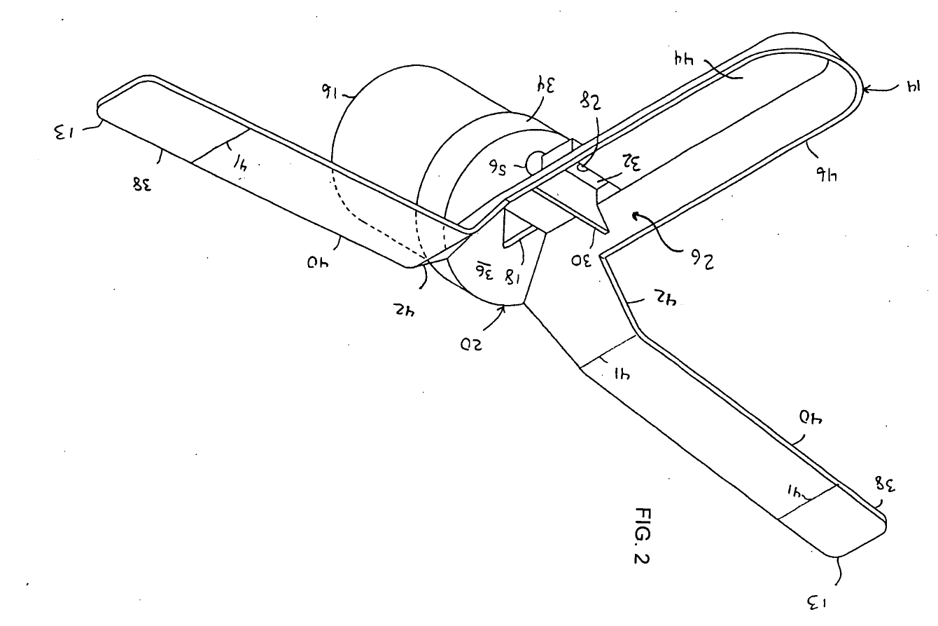

FIG. 2 shows the USCD 12 in an isolation view. The support arms 13 include opposite flat sections 38 that sit on top of the toilet bowl of toilet seat (see FIG. 1). Intermediate sections 40 can be bent slightly at an angle of about 15 degrees from horizontal lowering the collection section 14 and attachment section 20 below the top of the toilet bowl. Opposite support arm center sections 42 are angled approximately 45 degrees from horizontal and attach to the attachment section 20.

In one embodiment, the support arms 13 extend approximately 13 inches from the attachment section 20 to the ends and are approximately 1.5 inches wide. The length of the support arms 13 are sized to sit on top of any conventional toilet, however, the length can be varied for other toilet bowl widths. The support arms 13 can be made out of any material with enough strength to span the toilet and hold the USCD 12 and any urine samples. For example, a cardboard or plastic material can be used. The support arms 13 can include perforations 41 that are used to break the support arms 13 apart after use. This allows the USCD 12 to be more easily disassembled for inserting into a garbage can.

The angles of support arm sections 40 and 42 are set so the sample cup 16 sits at an angle above the conventional water level in the toilet bowl. However, for toilets that may have a higher water level, the angles of support arm sections 40 and 42 can be manually manipulated to sit higher in the toilet bowl. Alternatively, the support arms 13 can be made during manufacture with different angles for different toilet bowl water levels.

The collection section 14 includes a flat bottom section 44 and an upwardly and outwardly rising sidewall 46. The collection section 14 in one embodiment slopes upwardly from the attachment section 20 at an angle of approximately 15 degrees above horizontal. This further promotes the urine to flow downward toward opening 18. The collection section 14 in one example is approximately 4.5 inches long and 1.5-2 inches wide.

The collection section 14 is located directly under and in front of the urinary excretion point of the user. If for any reason the collection section 14 is not sitting in the proper location to catch the urine of the user, the support arms 13 can be repositioned on the toilet bowl and the arms bent to accommodate different anatomical characteristics of the urine sample provider.

The collection section 14 includes a flow divider 26 that allows collection of the urine “mid-stream” flow by redirecting a first portion of the urine flow out a bottom hole 28 and into the toilet bowl. The hole 28 is located in a chamber 32 that is approximately 2 cubic centimeters (ccs) in volume. As a user first starts to urinate, a first portion of the urine flows from the collection section 14 into chamber 32. The initial urine flow passes slowly out of chamber 32 through hole 28 and into the toilet.

The rate that the urine drains from hole 28 is much slower than the typical rate that urine flows into the chamber 32 from a user. Therefore, as the user continues to urinate, urine fills up and overflows chamber 32 and flows further downward toward opening 18 in the attachment section 20. The urine flowing into opening 18 is then collected in sample cup 16. This mid-stream flow sample is therefore cleaner and a more representative sample of the urine in the bladder compared with the first portion of the urine flow captured in chamber 32 and discharged out of hole 28.

A dam 30 can optionally be used to further prevent the initial urine flow from reaching the sample cup 16. The dam 30 extends upwardly from the back of the chamber 32 and in front of the opening 18. The dam 30 does not extend upwards as high as wall 46 of the collection section 14 so urine will not overflow over the wall 46. However, the dam 30 is high enough to further restrict the initial urine flow from flowing over the chamber 32 and into opening 18. If the purity of the urine is not of utmost importance, then another embodiment of the invention can be provided that does not provide the flow divider 26 or the dam 30 in collector section 14.

The attachment section 20 includes a rearwardly sloping back wall 36. The back wall 36 in combination with the angles of the middle sections 42 create a larger collection area that funnels the received urine into the opening 18. The attachment section 20 also includes a cap section 34 that receives the sample cup 16. Another unique feature is the ability of the sample cup 16 to be easily attached and detached to the cap section 34. The cap section 34 also includes an overflow hole 56 used for urine that overflows sample cup 16. The overflow hole 56 will be described in further detail below.

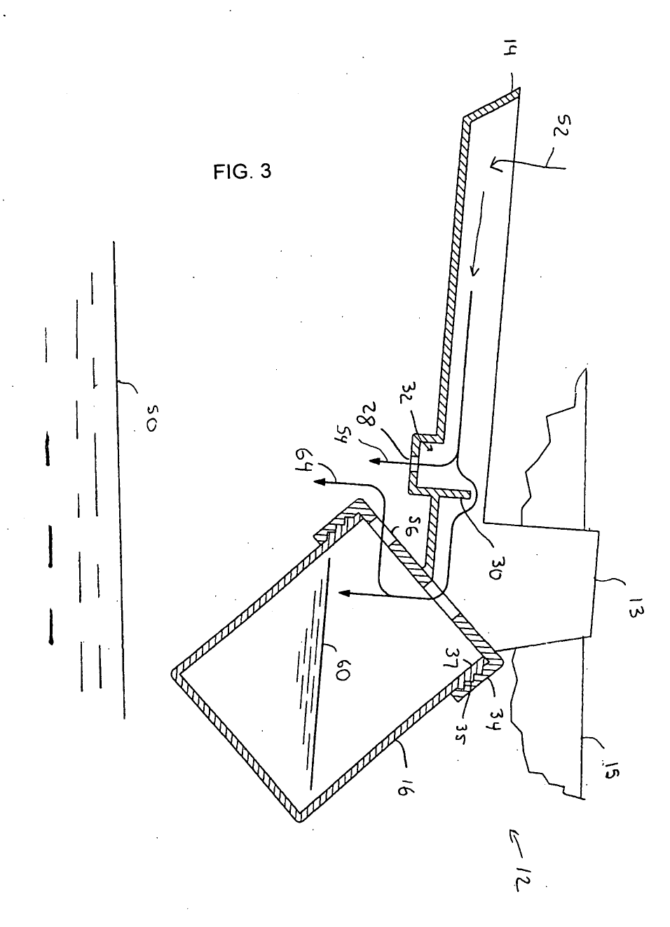

FIG. 3 is a side-sectional view of the USCD 12. A urine flow 52 is received by the collection section 14. The collection section 14 directs the initial urine flow into the chamber 32. An initial urine flow 54 flows into chamber 32 and through hole 28 into the toilet 14. As the chamber 32 fills up, the additional midflow urine rises above the dam 30 and flows through hole 18 and into sample cup 16.

The overflow hole 56 is located on the bottom side of the cap section 34 directly underneath the collection section 14. In cases when there is large amounts of urination, the urine 60 in sample cup 16 increases to the top of the left edge of the sample cup 16. Additional excess urine overflows through the overflow hole 56, over the left edge corner of the cap section 34, and into the water 50 in toilet bowl 14. Since the sample cup 16 is positioned at an angle, urine 64 will overflow through overflow opening 56 before the sample cup 16 is completely full. Thus, when the sample cup 16 is moved to an upright position, the urine 60 will only fill a portion of the sample cup 16.

As mentioned above, in one example, the sample cup 16 is held at an approximately 45 degree angle by the cap section 34 when sitting in the operational position inside the toilet bowl. However, the angle of the sample cup 16 can be larger or smaller angle according to what proportion of the sample cup 16 is desired to be filled with urine before excess urine overflows into the toilet.

The cap section 34 includes threads 35 on an inside wall that are used for threadedly engaging with a top end 37 of the sample cup 16. Thus, the sample cup 16 in this embodiment is screwed into the cap section 34. However, in alternative embodiments, the cap section 34 can be sized so that the sample cup 16 is slide or snapped into place in cap section 34. In the snapped embodiment, clips (not shown) may be located in the attachment section 34 for clipping onto the top end of the sample cup 16. In the force fit embodiment, the attachment section 34 may be slightly oval in shape to press in the sides of the sample cup 16. After the urine sample is given, the sample cup 16 is disconnected from USCD 12 and saved. In the embodiment shown in FIG. 3, the sample cup 16 is screwed out of the cap section 34. In other force fit of snap in embodiments, the sample cup is slid out of the cap section 34.

The USCD 12 can be made out of any of several type of plastic, elastomer, composite, coated paper, wire frame, or metal. In one example, the USCD 12 is made out of a coated paper or thin semi-rigid plastic material that is discarded after one use. Alternatively, the USCD 12 can be made out of a material that can be sanitized for reuse with other sample cups. In this type of embodiment, the USCD 12 may be made out of metal, heavy plastic or other non-porous rigid materials that can withstand the temperatures and high water forces used with heavy sanitization.

The USCD 12 can be used wherever urine samples are taken such as: medical clinics; doctor's office; hospitals; nursing homes and drug testing businesses, etc. The USCD can be one integral piece or several pieces attached together. The different sections may also be made from different materials. For example, the support arms 13 may be detachable from the attachment section 20 and the collection section 14. This would allow different pieces to be reused.

The USCD 12 has several unique benefits. It facilitates the collection of a urine sample by being hands-free. It has the capability to catch a “mid-stream” flow. The mid-stream flow is sometimes important, since the initial urine excretion may be contaminated by other substances. The USCD 12 can be a disposable one-time-use product and has a removable sample cup 16 attached directly to the rest of the USCD 12. The compact size and design also reduces the quantity of medical waste and is easily stackable for shipping multiple components at the same time.

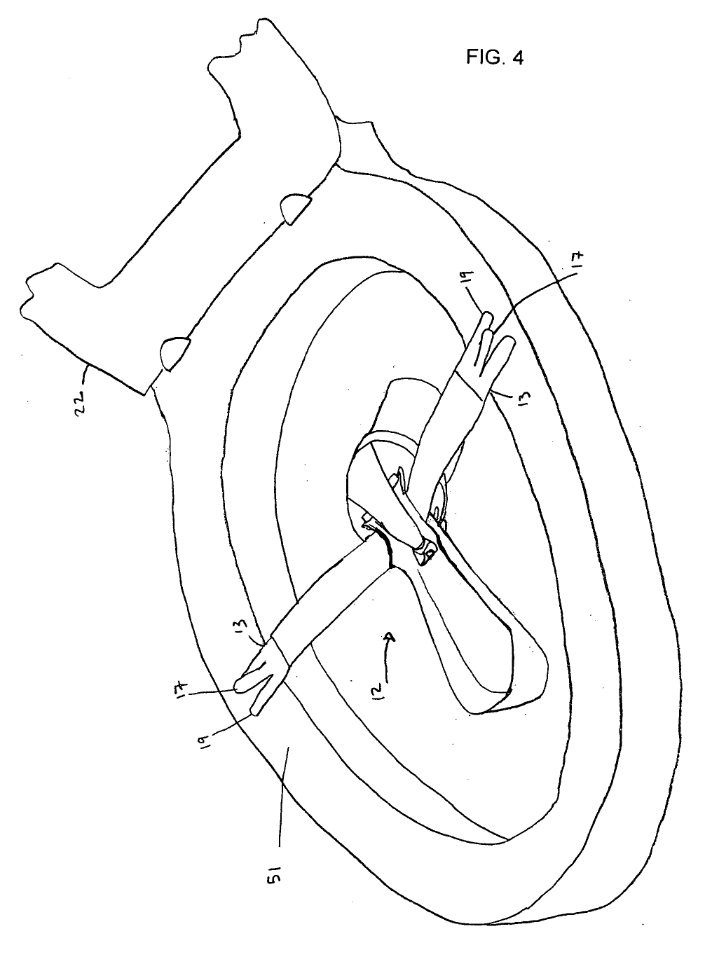

FIG. 4 is a second embodiment of the USCD 12. In this embodiment each support arm 13 includes an upper fixating tab 17 and two lower fixating tabs 19. The lower fixating tabs 19 contact the toilet rim 51 to secure the USCD 12. The upper fixating tab 17 is preferably made out of a flexible material such as plastic or cardboard. The flexible upper fixating tab 17 preferably flexes when making contact with a lowered toilet seat 22. The contact between the upper fixating tab 17 and the lowered toilet seat 22 further secures the USCD 12. The USCD 12 is thereby prevented from being unintentionally slid across toilet rim 51.

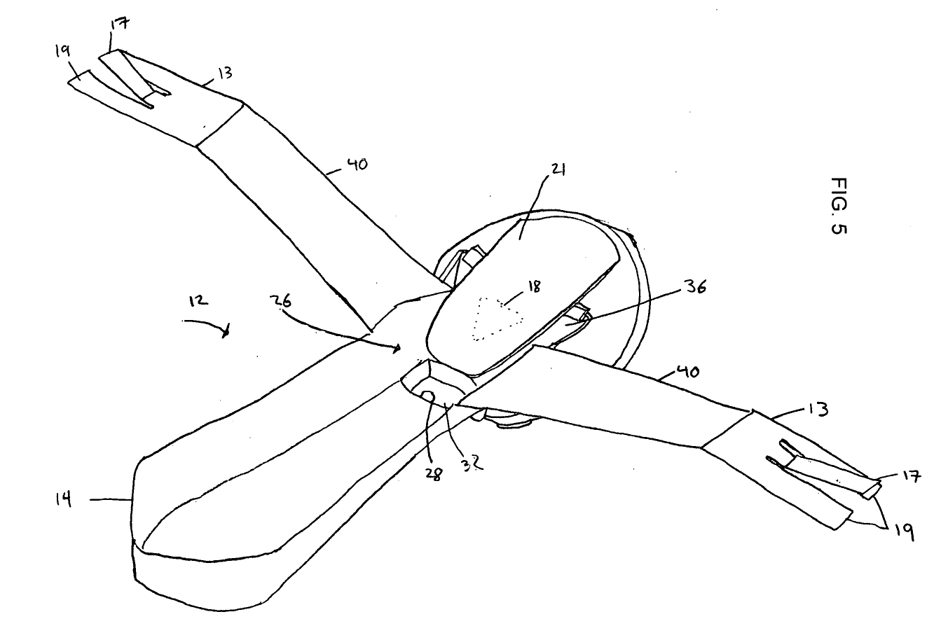

FIG. 5 is the second embodiment of the USCD 12 in an isolation view. The collection section 14 of the USCD 12 includes a flow divider 26 that allows collection of the urine “mid-stream” flow by diverting a first portion of the urine flow out a bottom hole 28 and into the toilet bowl 15. The hole 28 is located in a trough-shaped chamber 32 that is approximately 2 cubic centimeters (ccs) in volume. As a user first starts to urinate, a first portion of the urine flows from the collection section 14 into chamber 32. The initial urine flow passes slowly out of chamber 32 through hole 28 and into the toilet.

The attachment section 20 includes a rearwardly sloping back wall 36. The back wall 36 creates a larger collection area that funnels the received urine into the opening 18. The back wall 36 may be bubble-shaped to improve the collection property of the back wall 36.

A hood section 21 may be fixated to the rearwardly sloping back wall 36. The hood section 21 provides at least two functions. First, the hood section 21 prevents a misaimed flow of urine from directly entering the opening 18. A misaimed stream of urine is directed to the flow divider 26 before reaching the opening 18. Second, like the back wall 36, the hood section 21 creates a larger collection section. The hood section 21 may also be bubble-shaped like the back wall 36.

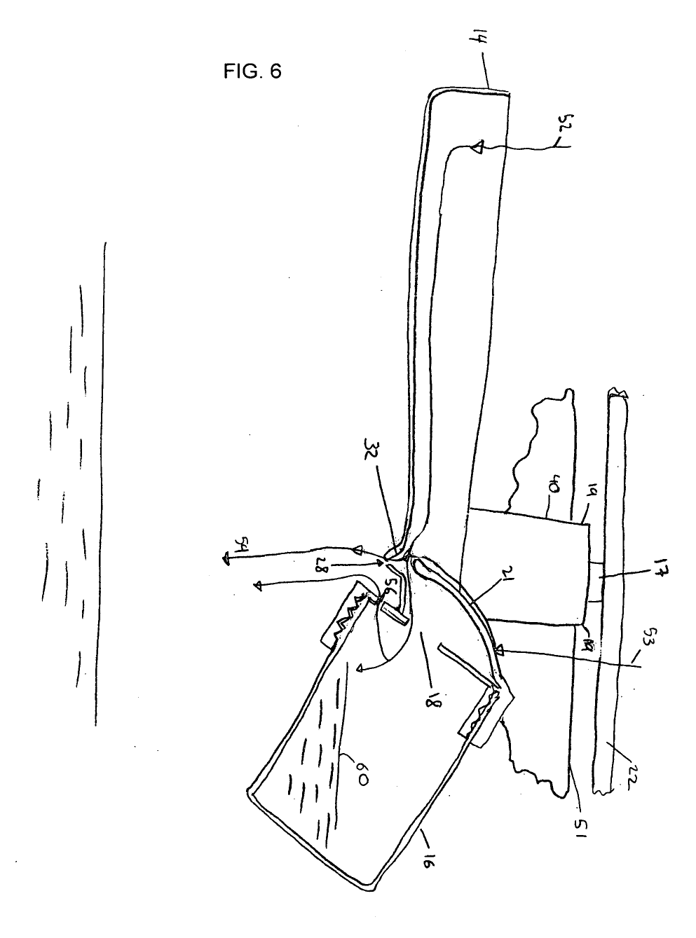

FIG. 6 is the second embodiment of the USCD 12 in a side-sectional view. A urine flow 52 is received by the collection section 14. The collection section 14 directs the initial urine flow into the chamber 32. An initial urine flow 54 flows into chamber 32 and through hole 28 into the toilet 14. As the chamber 32 fills up, the additional midflow urine flows through hole 18 and into sample cup 16.

A misaimed flow of urine 53 is received on the hood section 21. The hood section 21 prevents the misaimed flow of urine 52 from directly entering the sample cup 16 through the opening 18. The hood section 21 may be curved to minimize splashing during the diversion. The misaimed flow of urine 52 is diverted by the hood section 21 towards the collection section 14. If the misaimed flow of urine 52 is also an initial flow of urine, the misaimed flow of urine 52 flows into the chamber 32 and through the hole 28 into the toilet 14. As the chamber 32 fills up, the misaimed flow of urine 52 that is also midflow urine flows through the hole 18 and into the sample cup 16.

The force of the misaimed flow of urine 52 will not change the position of the USCD 12 on the toilet rim 51 at least because of the fixating tabs 17 and 19. The lower fixating tabs 19 are in contact with the toilet rim 51. The upper fixating tab 17 is in contact with the closed toilet seat 22. The effect of the contact between the fixating tabs 17 and 19 and the toilet rim 51 and toilet seat 22 is that the USCD 12 is wedged into a secure position.

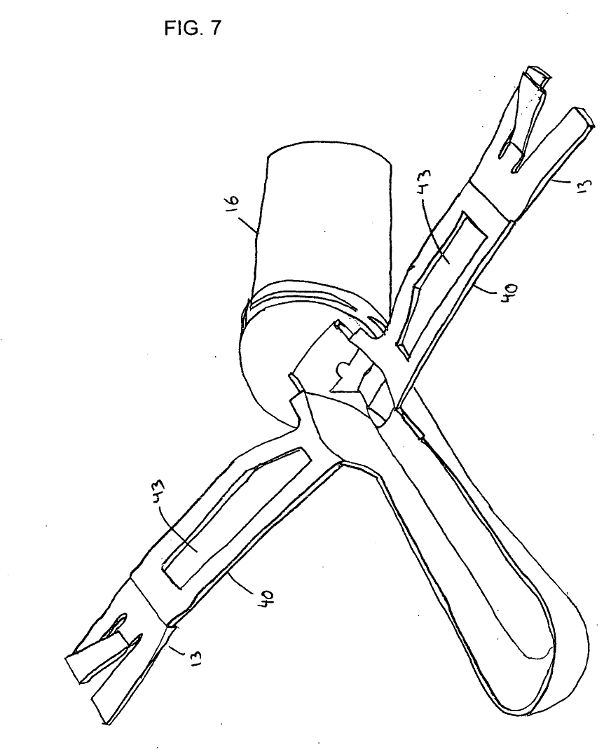

FIG. 7 is a third embodiment of the USCD 12 in an isolation view. In this embodiment intermediate sections 40 of the support arms 13 include openings 43. The openings 43 in the intermediate sections 40 reduce material cost for the USCD 12.

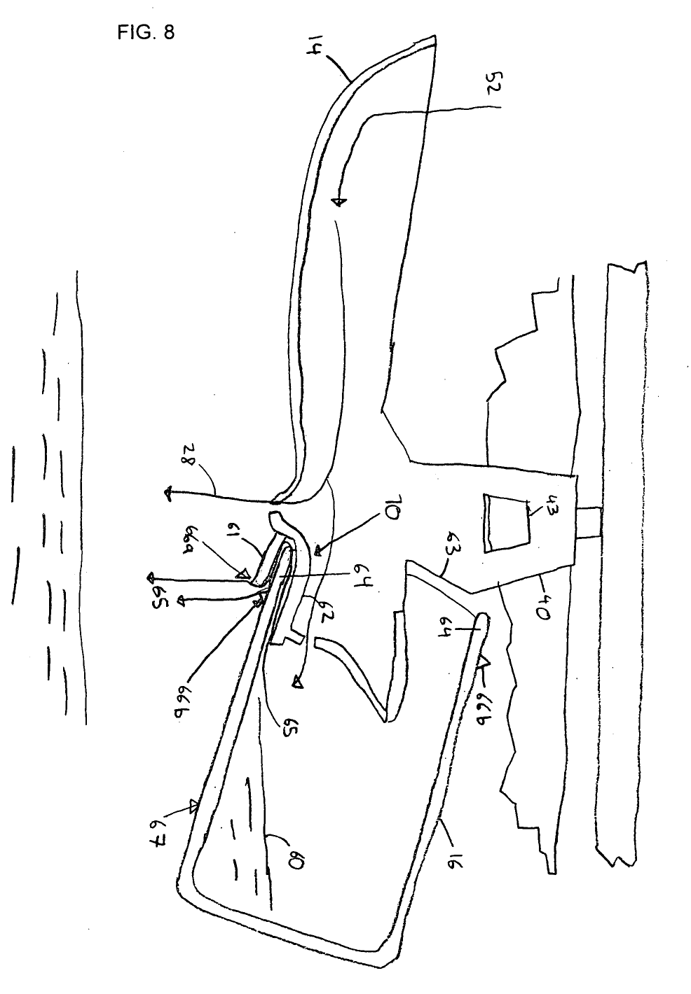

This embodiment of the USCD 12 does not include a cap section and can accept sample cups of many different sizes and shapes. The sample cup 16 is secured to the USCD 12 by inserting a rim of the plastic cup into the USCD 12. This feature will be described in more detail with reference to FIG. 8.

FIG. 8 is the third embodiment of the USCD 12 in a side-sectional view. The sample cup 16 detachably couples to the USCD 12 at the attachment section 70. The attachment section 70 includes a lower flange 61 and an upper flange 62. The rim 64 of the sample cup 16 is disposed between the flanges 61 and 62 thereby securing the sample cup 16. As the sample cup 16 fills with urine 60 the magnitude of normal forces acting on the rim 64 of the sample cup 16 is increased, thereby further securing the sample cup 16. Advantageously, the attachment section 70 obviates the need for a threaded sample cup of a predetermined diameter.

The attachment section 70 also acts as an overflow for the sample cup 16. When the urine 60 in the sample cup 16 rises above the rim 64, an overflow portion 65 flows between the sample cup 16 and the lower flange 61. The lower edge 66a of the lower flange 61 and/or the thread 66b of the sample cup 16 may act as a drip edge for the overflow portion 65. Advantageously the overflow portion 65 drips off these drip edges 66a and 66b and does not get on the outside 67 of the sample cup 16.

Each support arm section 40 also may include a cut out section 63. Cut out section 63 accommodates the rim 64 of the sample cup 16. The cut out section 63 allows a large sample cup 16 to be fully inserted into the attachment section 70 without the rim 64 being forcibly pressed against the support arm section 40.

The collection section 14 is shallower than other embodiments, which allows for improved stacking. The collection section 14 is approximately 0.5 inches deep. The shallow collection section 14 allows for the USCD 12 to be stacked with only approximately 0.1875 inches of space between two stacked USCDs.

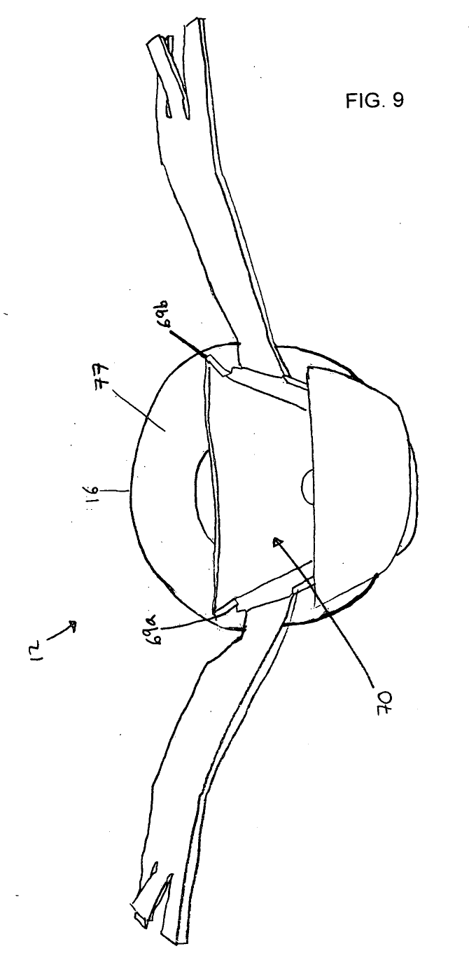

FIG. 9 is the third embodiment of the USCD 12 in a front isolation view. Stabilizers 69a and 69b optionally reinforce the function of the attachment section 70. The stabilizers 69a and 69b make contact with an inside wall 77 of the sample cup 16. The contact between the stabilizers 69a and 69b and the inside wall 77 further secures the sample cup 16.

Having described and illustrated the principles of the invention in a preferred embodiment thereof, it should be apparent that the invention may be modified in arrangement and detail without departing from such principles. We claim all modifications and variation coming within the spirit and scope of the following claims.

1. A urine collection device, comprising:
an apparatus configured to sit on a toilet and receive urine, the apparatus including a collection section directing received urine into a detachable sample holder and a flow divider that is structured to divert an initial portion of received urine into the toilet.

2. The urine collection device according to claim 1 further comprising a fixating section coupled to the apparatus, the fixating section structured to make contact with a toilet rim and a lowered toilet seat.

3. The urine collection device according to claim 2 wherein the fixating section comprises at least one lower fixating tab structured to make contact with a toilet rim and at least one flexible upper fixating tab structured to make contact with a lowered toilet seat.

4. The urine collection device according to claim 1 wherein the flow divider includes a cavity in the collection section that collects the initial portion of the urine and a hole at a bottom end of the cavity that directs the initial portion of the urine into the toilet.

5. The urine collection device according to claim 1 where the apparatus is structured to slidingly engage both an inside and outside sidewall of the sample holder.

6. The urine collection device according to claim 5 wherein the apparatus further comprises an upper flange that is structured to make contact with an inside portion of the sidewall and a lower flange that is structured to make contact with an outside portion of the sidewall.

7. The urine collection device according to claim 5 wherein the apparatus is further structured to act as a drip edge for an overflow from the sample holder.

8. The urine collection device according to claim 1 wherein the apparatus includes an opening in a front face that directs urine from the collection section into the sample holder.

9. The urine collection device according to claim 1 wherein the apparatus includes support arms that sit on the toilet for holding the sample holder in the toilet above a toilet water level.

10. The urine collection device according to claim 9 wherein the support arms comprise material-saving openings.

11. A urine collector, comprising:
a support apparatus for supporting the urine collector on a toilet;
a collection section integrally formed with the support apparatus; and
an attachment section for detachably coupling to a sample holder;
wherein the attachment section is structured so that a sidewall of an attached sample holder is disposed at a downwardly directed angle toward a back of a toilet.

12. The urine collector according to claim 11 wherein the disposition of the sample cup allows the urine collector to be used with differently sized toilet bowls without contact between the sample holder and either a toilet bowl or toilet water.

13. The urine collector according to claim 111 wherein the collection section is structured to extend towards a front end of the toilet so that the collection section and the attached sample holder counterbalance each other.

14. The urine collector according to claim 11 further comprising a fixating section coupled to the support apparatus, the fixating section structured to simultaneously make contact with a toilet rim and a lowered toilet seat.

15. The urine collection device according to claim 14 wherein the fixating section comprises at least one fixating tab structured to flex when in contact with a lowered toilet seat.

16. The urine collector according to claim 11 wherein the attachment section is structured to simultaneously contact both an inside and outside sidewall of the sample holder.

17. A urine sample collection device, comprising:
an apparatus configured to sit on a toilet bowl and receive urine, the apparatus directing the received urine into a detachable sample holder; and
a universal attachment section structured to slidingly engage differently sized detachable sample holders, the differently sized detachable sample holders having openings with different diameters.

18. The urine sample collection device according to claim 17 wherein the universal attachment section further comprises an upper flange that is structured to make contact with an inside portion of a sidewall of the sample holder and a lower flange that is structured to make contact with an outside portion of the sidewall of the sample holder.

19. The urine sample collection device according to claim 18 where the universal attachment section further comprises at least one stabilizer structured to contact the inside portion of the sidewall of the sample holder.

20. The urine sample collection device according to claim 18 wherein a magnitude of a normal force acting on the lower flange and caused by the outside portion of the sidewall increases when the sample cup is filled with urine.

21. A urine collection device, comprising:
support arms for supporting the urine collection device on a toilet;
an attachment section integrally formed with the support arms; and
a collection section integrally formed with the support arms for receiving and directing urine into a sample holder.

22. The urine collection device of claim 21 further comprising a fixating section coupled to support arms, the fixating section structured to make contact with a toilet rim and a lowered toilet seat.

23. The urine collection device of claim 21 wherein the attachment section is structured to slidingly engage an inside portion of a sidewall of the sample holder.

24. The urine collection device of claim 23 wherein the attachment section is further structured to act as a drip edge for an overflow from the sample holder.

25. The urine collection device of claim 21 further comprising a flow divider in the collection section, the flow divider structured to divert an initial portion of received urine into the toilet.
